(19) United States
(12) Patent Application Publication
(10) Pub. No.: US 2018/0182472 A1
(43) Pub. Date: Jun. 28, 2018

(54) METHOD AND SYSTEM FOR A MOBILE HEALTH PLATFORM

(71) Applicants: Kenzie L. PRESTON, Baltimore, MD (US); David H. EPSTEIN, Balltmore, MD (US); Mathew TYBURSKI, Baltimore, MD (US); Massoud HABZADEH, Towson, MD (US); United States of America, as Represented By The Secretary, Dept. of Health And Human Services, Bethesda, MD (US)

(72) Inventors: Kenzie L. Preston, Baltimore, MD (US); David H. Epstein, Baltimore, MD (US); Massound Habzadeh, Baltimore, MD (US); Matthew Tyburski, Baltimore, MD (US)

(21) Appl. No.: 15/580,975

(22) PCT Filed: Apr. 27, 2016
(86) PCT No.: PCT/US2016/029553

§ 371 Date: Dec. 8, 2017

Related U.S. Application Data
(60) Provisional application No. 62/186,983, filed on Jun. 30, 2015.

(51) Int. Cl. G16H 10/60; G06N 5/02; G06N 99/00

(52) U.S. Cl. CPC G16H 10/60; G06N 99/005; G06N 5/022

(57) ABSTRACT

Aspects of the present disclosure involve systems, methods, computer program products, and the like, for tracking, assessing and predicting human behavioral disorders in real time through a mobile device. In general, the mobile health platform involves tracking a geographical location of a user of the system through the mobile device, receiving environmental and user-provided information through the mobile device or from another source, and processing the received information. In one embodiment, the processing of the received information provides for a prediction of a future human behavior and such a prediction may be provided to the user's mobile device. For example, the information may indicate that a user of the mobile device is at risk for a particular human behavior and, as a result, a warning of the risk of the human behavior is transmitted to the user's mobile device.

### RELATED APPLICATIONS

This application claims priority under 35 U.S.C. § 119 from U.S. provisional application No. 62/186,983 entitled “MOBILE HEALTH PLATFORM,” filed on Jun. 30, 2015, the entire contents of which are fully incorporated by reference herein for all purposes.

### FIELD OF THE DISCLOSURE

Embodiments of the present invention generally relate to systems and methods for implementing a health platform utilizing a mobile device, and more specifically for analyzing environmental information and user-provided information to predict a potential human behavior and/or provide a warning to the user of the mobile device of a potential human behavior.

### BACKGROUND

Advances in genetics have provided a vast understanding of the genetic influences on human behavior, such as drug use and addiction. However, little is known about non-genetic influences, known collectively as the environment, on general human behaviors. For example, real-time assessment of exposure to, and responses to, drugs and psychosocial stress and assessment of how such exposures and responses vary across geographical locations is typically not examined and may be useful in understanding the causes of certain kinds of human behavior. With such environmental-specific analysis, studies of the genetics of human behavior may become more sensitive to the effects of genes whose roles in behavior are subtle or environmentally specific.

### SUMMARY

One implementation of the present disclosure may take the form of system for providing an intervention notice to a user of a mobile device. The system may comprise a network communication port for receiving a transfer of data from a mobile computing device, the received data from the mobile computing device comprising at least one indication of a geographic location of a user of the mobile computing device, a database configured to store the received data from the mobile computing device, and a computing device. The computing device may include a processing device and a computer-readable medium with one or more executable instructions stored thereon, wherein the processing device of the computing device executes the one or more instructions to perform certain operations. Such operations performed by the processing device may include receiving environmental risk mapping information associated with the user of the mobile computing device and executing predictive analytics on the correlated environmental risk mapping information with at least one indication of the geographic location of the user of the mobile computing device, the predictive analytics comprising a predicted behavior of the user of mobile computing device. Further, the processing device may transmit an automated decision to the mobile computing device through the network communication port, the automated decision configured to cause the mobile computing device to generate an intervention indicator for the user of the mobile computing device to alter the predicted behavior of the user.

Another implementation of the present disclosure may take the form of a computer-implemented method for an automated assessment of the momentary status of a user. The method may include the operations of receiving a transfer of data from a mobile computing device associated with a user through a network connection, the received data from the mobile computing device comprising at least one indication of a geographic location of the user and storing environmental risk mapping information and the received data from the mobile computing device in a database. In addition, the computer-implemented method may include executing predictive analytics on the environmental risk mapping information with at least one indication of the geographic location of the user to generate a future prediction for the status of the user based on a machine learning model and transmitting an automated decision to the mobile computing device through the network, the automated decision configured to cause the mobile computing device to generate an intervention indicator for the user to alter the predicted status of the user.

Yet another implementation of the present disclosure may take the form of one or more non-transitory tangible computer-readable storage media storing computer-executable instructions for performing a computer process on a machine. The performed computer process includes the operations of receiving initial user data from a user of a human behavior intervention system, storing the initial user data in a user database with an environmental risk mapping information obtained from a third party database, and receiving a transfer of data from a mobile computing device through a network connection, the received data from the mobile computing device comprising at least one indication of a geographic location of a user of the mobile computing device. The process may also include correlating the received environmental risk mapping information with at least one indication of the geographic location of the user of the mobile computing device, executing predictive analytics on the correlated environmental risk mapping information with at least one indication of the geographic location of the user of the mobile computing device, the predictive analytics comprising a predicted behavior of the user of mobile computing device, and transmitting an automated decision to the mobile computing device through the network communication port, the automated decision configured to cause the mobile computing device to generate an intervention indicator for the user of the mobile computing device to alter the predicted behavior of the user.

### BRIEF DESCRIPTION OF THE DRAWINGS

FIG. 1 is a schematic diagram illustrating a general process of a mobile health platform system.

FIG. 2 is a schematic diagram illustrating a process for developing a database in relation to a mobile health platform system.

FIG. 3 is a schematic diagram illustrating a process for a data processing analytics sequence utilized with a mobile health platform system.

FIG. 4 is a schematic diagram illustrating a pre-processing sequence for a mobile health platform system.

FIG. 5 is a schematic diagram illustrating a behavioral statistics sequence for a mobile health platform system.

FIG. 6 is a schematic diagram illustrating a machine learning sequence for a mobile health platform system.

FIG. 7 is a schematic diagram illustrating a machine implemented sequence for a mobile health intervention on a mobile health platform system.

FIG. 8 is a schematic diagram illustrating a machine learning model database for a mobile health platform system.

FIG. 9 is a diagram illustrating an example of a computing system which may be used in implementing embodiments of the present disclosure.

### DETAILED DESCRIPTION

Aspects of the present disclosure involve systems, methods, computer program products, and the like, for tracking, assessing and predicting human behavioral disorders in real time through a mobile device. In general, the mobile health platform involves tracking a geographical location of a user of the system through the mobile device, receiving environmental and user-provided information through the mobile device or from another source, and processing the received information. In one embodiment, the processing of the received information provides for a prediction of a future human behavior and such a prediction may be provided to the user's mobile device. For example, the information may indicate that a user of the mobile device is at risk for a particular human behavior and, as a result, a warning of the risk of the human behavior is transmitted to the user's mobile device. In one embodiment, the mobile device may provide some indication of the perceived risk to the user of the device.

In a further embodiment, a machine learning process may be employed within the mobile health platform to improve and tune the prediction of future human behavior. Thus, information obtained from one or more users of the mobile health platform may be provided to the machine learning process, as well as the accuracy of provided predictions of the human behavior. Through multiple iterations of the processing and application of information to provide a prediction, the mobile health platform may become more and more accurate over time. Further, the predictions of the mobile health platform may adjust to new environmental information or types of users through the machine learning process. In one embodiment, the prediction of the mobile health platform may be specific to an individual user. In other embodiments, groups or other subsets of the users of the system may be analyzed to provide a prediction.

In one particular implementation of the mobile health platform, analysis and prediction of human behaviors may be applied for individuals suffering with drug addiction. In practice, the mobile health platform provides an automated prediction for a future risk of drug use in real time as an individual goes about their daily life. Such a warning to an individual of a pending negative event may be received through a mobile device carried or otherwise corresponding to the user. However, while the present disclosure was initially developed as a mobile intervention for drug addition, its application is not limited to drug addiction or other risky behaviors. Rather, the mobile health platform may be applied to any endeavor that includes human behaviors, from risk management of an organization to advertisement for purchasing of a good. In general, the platform is envisaged to be utilized and applied to any intervention requiring behavioral change.

In particular, the mobile health platform described herein can be applied to tracking mental health disorders and generating an automated prediction for the risk of a possible negative behavioral episode. The automated prediction could be delivered to either: i) the user, ii) a health professional, iii) both or iv) an other actor that could instigate change in the user. The following is a non-exclusive list of possible behavioral disorders that the mobile health platform could be used to change: 1) Attention Deficit Hyperactivity Disorder, 2) Drug abuse, 3) Alcohol use/abuse/Alcoholism, 4) Gambling addiction, 5) Alzheimer's Disease, 6) Binge eating and eating disorders, 7) Bipolar disorder, 8) Depression and depressive disorders, 9) Generalized anxiety disorder, 10) Mood disorders, 11) Panic disorder, 12) Post-traumatic stress disorder, and 13) Cigarette smoking. However, it should be appreciated that the systems and methods described herein may be utilized for any purpose in providing a notification to a user of a mobile device.

One advantage of accurate assessment of environmental exposure (to stressors, drug availability, or drugs themselves) is minimizing the delay between exposure and reporting. The tools proposed and discussed herein, such as personal digital assistants (PDAs) and/or global positioning system (GPS) units, are those which users can carry with them during their daily routines, enabling them to report stress and drug use as they occur. Proximate self-reported data collection may occur in real time and be compared to data from standard retrospective self-report methods and to biological measures, all from the same users. Further, obtaining real-time geographic-location data allows for evaluation of the roles of different neighborhoods or areas, which will be compared to standard fixed demographic indicators such as current address.

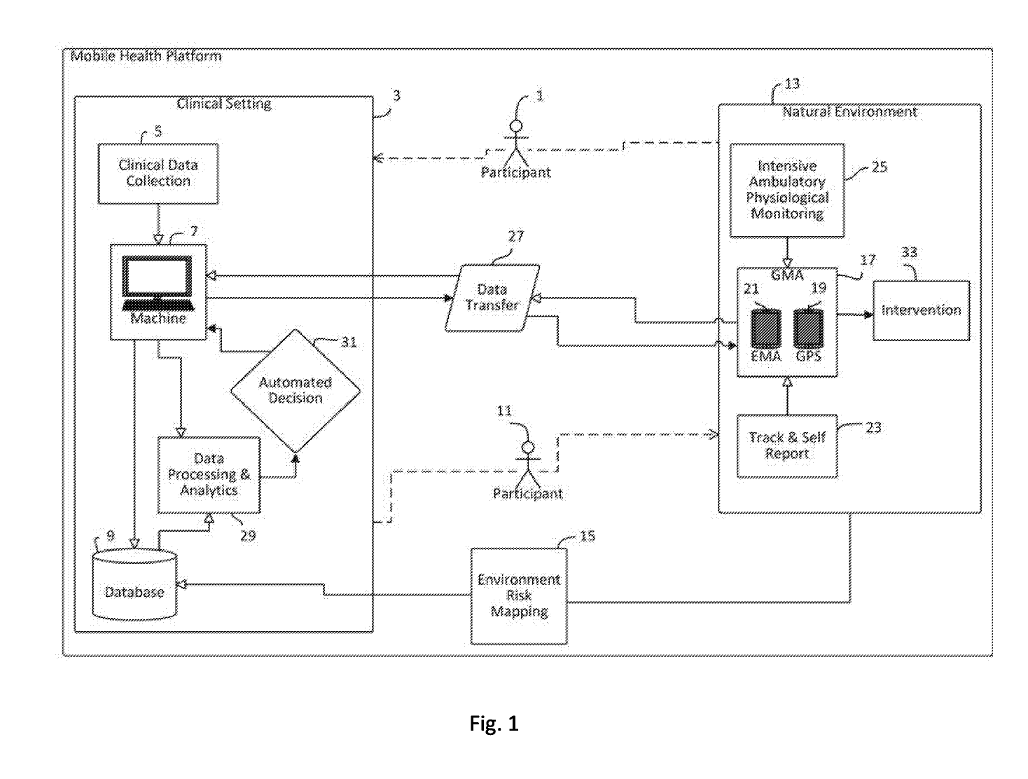

FIG. 1 is a schematic diagram illustrating a general process of a mobile health platform system. In general, the operating process of the mobile health platform includes the transmission of information between various components, devices, and machines of the platform. For example, a mobile device 17 may be utilized to track a user's position, receive information from the user, and provide one or more messages or other indicators to the user. A server 7 or other network device may receive information from the mobile device 17 and process the information as described below. A database 9 may be in communication with the server 7 to store received information and/or instructions executed by the server to perform one or more of the operations described below. Further, although particular components of the mobile health platform are described herein, it should be appreciated that any number of additional components may be included in the system to facilitate the collection, transmission, and processing of the information discussed without deviating from the operation of the system described herein. For example, the mobile device 17 may communicate wirelessly with the server 7 through a telecommunications network comprising any number of telecommunication devices, connections, routers, and the like. Thus, although some components of the mobile health platform are discussed herein, other components may be included as understood by those of ordinary skill in the art.

To begin the process of the mobile health platform, a study user or clinic patient 1 provides user information to a clinical setting 3. “Clinical setting” may include any device 7 where health and behavioral data 5 may be received and collected by a machine and stored in a database 9. For example, the machine 7 may be a server, laptop, personal computer, tablet, or any other computing device that receives information 5 about the user 1. Further, the machine 7 may be in communication with the database 9 for storing the received information 5, among other received information. The user information may be provided to the machine 7 from the user himself, from another computing device (such as a mobile device), or from a user or operator of the machine.

In one embodiment of the process of the mobile health platform, the machine 7 may also collect data on the user's natural environment 13 through a data transfer 27 to assess exposure to environmental risks or behavioral triggers. Such information may be provided to the machine 7 through a mobile device 17 associated with the user 1. Further, additional data on the user's natural environment may be transmitted and stored as digital geo-referenced environmental-risk maps 15 in the database 9. The environmental-risk maps 15 are constructed from interpolated numbers that can represent 1) independent observers' ratings of risk (or events and/or measures contributing to risk) in the geographical regions where the user spends time and/or moves (i.e., activity space) or 2) user-entered locations of the sites of negative events. The maps 15 can also be derived from third party databases of public information, such as crime data, or commercial density and locations, such as liquor stores, bars and/or shopping centers, income information (such as from tax data), and the like. For example, areas listed as “high crime” areas from a third party database may be included in the environment risk maps 15 and provided to the database 9 for storing. In another example, databases available through a public network, such as the Internet, may be mined for information to provide to the database 9 of the mobile health platform. Areas or other information of the environment risk mapping 15 may be used as described further below to determine when a user 1 is in a risky situation and an intervention or warning is provided to the user. The numbers used in the environmental risk maps 15 can be “presence only” indicators or can be dichotomous, categorical, or continuous measures.

In general, after the initial/periodic collection of data 5 at the clinic machine 7, the user (indicated in FIG. 1 as user 11, although user 1 and user 11 may be the same actor) returns to his or her natural environment 13 to go about daily-life activities. During this time, a technique referred to herein as Geographical Momentary Assessment (GMA) is used to collect information about the user's behavior in the natural environment on a handheld device 17. The GMA is a combination of a Geographical Positioning System (GPS) 19 and Ecological Momentary Assessment (EMA) 21 that can be done on one or more mobile devices 17. For example, GMA data may include movement (i.e., track) information (GPS) 19 and user self-reports regarding moods and behaviors (EMA) 23. A GPS unit 19 in communication with the mobile device 17 collects time-stamped movement (i.e., track) information about a user's location. An EMA unit 21, which can be integrated with or separate from the GPS unit, allows the user to report moods and behaviors as they occur 23. For example, a program may be executed by the mobile device 17 that provides an interface through which a user of the mobile device provides the EMA information 23. EMA data 23 may be collected in at least two ways: 1) randomly timed prompts to complete on-screen questionnaires, and 2) user-initiated event reports whenever a behavior of interest occurs (e.g., smoking, binge eating, or any other behavior of interest to the mobile health platform).

In addition, intensive ambulatory physiological monitoring 25 can be added to the GMA through one or more components of the mobile device 17. For example, one or more sensors, such as accelerometers, may be in communication with the mobile device 17 to collect physiological information about a user in real time in the natural environment. In another example, a blood pressure device may be worn by the user 11 and the user's blood pressure may be monitored and included in the EMA 21. The above described collected data is transferred 27 to the clinical machine 7 and stored in the database 9. In general, the data transfer can be done: 1) directly via a hardwire connection in the machine 7 or an accompanying network, 2) remotely via Bluetooth (or another wireless data transmission process), 3) through an automated data dump via secure html over the Internet when the user is in their natural environment, 4) via an automated cloud connection when the user is in their natural environment, and the like.

In one embodiment of the mobile health platform system, the database 9 may contains: 1) user data collected in the clinical setting 5 from the user 1, environmental risk maps made independently of or concurrently with the user 15, and GMA data (EMA data 21, GPS location data 19, ambulatory physiological monitoring data 25, user-reported data 23, etc.) collected from the user in the natural environment 17. The data received at the clinical setting 3 may be processed and analyzed by the machine 17, as indicated in box 29 of FIG. 1. The output of the processing and analyses 29 is an automated decision 31 predicting potential future events that are behaviors of clinical interest to the user 11. The machine 7 next transmits the automated decision through the data transfer 27 back to the mobile device 17 of the user by any of the methods mentioned above. In some instances, the mobile device 17 displays the prediction of a future event on the screen while the user is in their natural environment 31 to invoke a reaction by the user 11. The display of the automated prediction is referred to as the intervention 33. The intervention 33 can then be coupled to other information transmitted to the user via the mobile device 17 that helps the user cope with, and potentially prevent, a negative event.

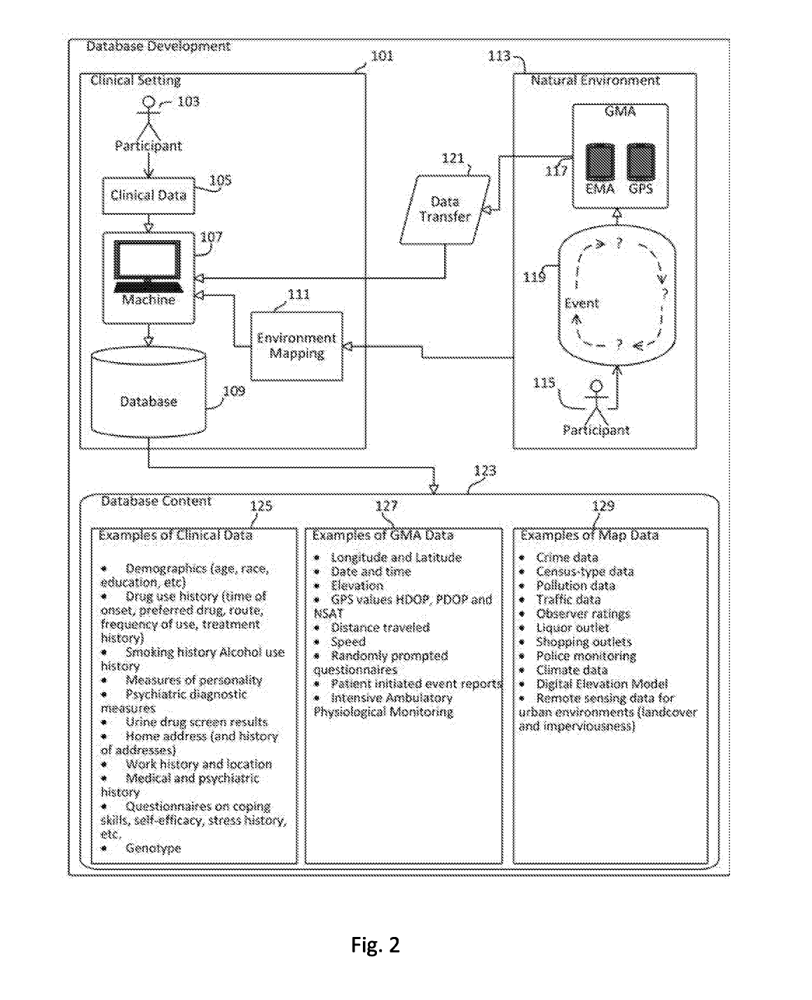

FIG. 2 is a schematic diagram illustrating a process for developing a database 109 in relation to a mobile health platform system. The database 109 of FIG. 2 may be similar to the database described above with reference to FIG. 1. As described, information is obtained by the machine 107 of the clinical setting 101 and stored in the database 109. In particular, clinical data 105 collected concerning a user 103 through the machine 107 may be stored in a database 109. Environmental risk mapping 111 is obtained from one or more third party or public databases and completed on the machine 107 and also stored on a database 109. In a natural environment 113, a user 115 may carry a GMA unit or mobile device 117, which collects data on the user's movement and records the user's responses to questions about events 119. The data collected by the GMA unit 117 are transferred 121 back to a machine 107 and stored on a database 109. This data transfer 121 may occur over any communication medium known or hereafter developed, including but not limited to, wired communication over a network, wireless communication over a network, a combination of wired and wireless networks, mobile hard drive, etc. Box 123 of FIG. 2 illustrates some examples of the content of the database 109 organized into three types of directories or sub-databases: 1) clinical data 125, 2) GMA data 127 and 3) environmental data collected independently of the user 129. Examples of these three categories of data stored in a database are provided in list 125, list 127 and list 129 and in relation to drug addiction. In general, however, the information stored in the database 109 may include any information associated with a human behavior and may be organized in any manner.

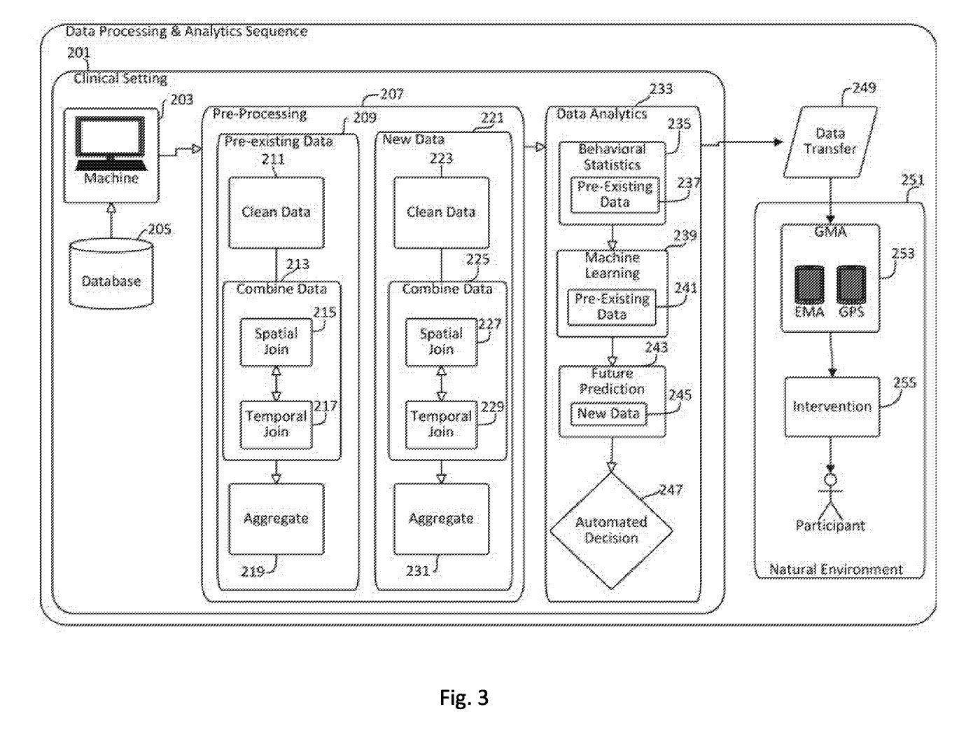

FIG. 3 is a schematic diagram illustrating a process for a data processing analytics sequence 201 utilized with a mobile health platform system. Through the process illustrated in FIG. 3, a prediction of behavior may be generated by a machine 203 or computing device and delivered to a user via a GMA unit 253 or mobile device to act as an intervention 255 event for the user. The process starts when the machine 203 (such as a network server or other computing device) is used to access data stored in a database 205. One example of the data included in the database 205 is illustrated in FIG. 2. The machine 203 executes a Pre-Processing operation 207, which is broken into two types of data flows. The first data flow 209 deals with pre-existing 209 or previously collected data that is in the database 205 (independently of new data 221 collected from the user in real time). The second data flow 221 is for newly received or stored data in the database 205. Both Pre-existing 209 and New Data 221 processing flows follow a similar processing sequence. In general, the difference between the two sequences is that they may also include data from other users of the mobile health system and/or other outside sources to be combined with a specific user's data. Pre-existing Data 209 may also be used to develop automated data analytics that are applied to New Data as they come in via real-time transfer. New Data 209 are data collected from a user and transferred (through data transfer 27, 121) to the database in real time from a user, a user's mobile device, or other third party database or computing device.

In the pre-processing 207 step for pre-existing data 209, the machine 203 identifies raw data that may be of poor quality and flags them so that they will not be used in the processes of the system. In one embodiment, identification of poor-quality data is completed mathematically by computer-implemented statistical operations executed by the machine 203. The data that are not identified as of poor quality are combined 213 by the machine 203. In general, combining the data can be completed as a spatial join or intersect 215 with other spatial data or as a temporal join 217 with non-spatial data that have a timestamp. These two operations, as is possible with most of the operations described herein, are interchangeable in their order of operations. The spatial join or intersect 215 is completed with the GMA data, for example, by using the longitude and latitude collected by the GPS component of the mobile device 17. The GPS data are then spatially overlaid on digital environmental-risk maps by the machine 203. For example, the GPS data are used to sample the environmental-risk maps at the relevant longitudes and latitudes. The output is a new GIS shape file or text file (i.e., .txt or csv file) with the GPS data combined to the environmental-risk map data 15. The temporal join 217 is completed, for example, by intersecting the data collected by the GPS component with time stamps of data that are not geographically referenced. The term “joining by timestamp” may include combining or fusing different information about a user 11 (i.e., collected by different devices or sensors) into comparable increments in time. For example, the devices used for EMA, GPS, and intensive ambulatory physiological monitoring 25 can collect data simultaneously, but at different temporal frequencies: the EMA data might be collected sporadically, reflecting a handful of events per day, while the GPS data might consist of multiple events per minute or hour, and the physiology data can be collected at the sub-second level. In some instances, the data cannot be analyzed without being joined together due to the difference in temporal collecting of the data. The joining by timestamp described herein allows the EMA 21 to be connected to GPS 19 and/or the physiological sensors 25, and the GPS timestamp allows the EMA and physiological sensors to be linked spatially to environmental-risk maps 15. Once the data are joined together, a final Pre-Processing operation is utilized to aggregate the joined data to comparable spatial or temporal units that can be fed into the computational data analytics. This may include aggregating high-frequency data by averaging the joined data (i.e., by space and/or time) over larger incremental instances, such as one replicate every 10, 20, or 30 minutes. Aggregating also produces consistent temporal data replication for randomly varying data, such as EMA 21 or speed-based GPS 19 data collection.

In the pre-processing 207 step for New Data 221, the operations are implemented by a machine 203 after real-time GMA data are transferred 27, 121 from the mobile device 17. The New Data are accessed by a machine 203 and any poor-quality data are removed 223 from the dataset, similar to above. Identifying poor-quality data 223 may be completed mathematically by computer implemented pre-existing statistical operations that were developed as discussed above with reference to step 211. The New Data are then combined 225 by spatial 227 and temporal 229 joining methods consistent with the same processes as in operation 213 described above. The New Data are then aggregated in 231 so that they are in units consistent with the aggregated data from step 219.

After the data are Pre-Processed 207, they are fed into a Data Analytics 233 sequence of processing steps. The outcome of the Data Analytics 233 processing sequence is an automated or manual decision (i.e., based on machine learning) that predicts a future event of interest for a user. The first step in the sequence 233 is to run Behavioral Statistics 235 to detect reliable relationships among GMA entries (i.e., “how much are you craving drugs?”), intensive ambulatory physiological data, and exposure to environmental risks. An example of a specific type of Behavioral Statistic 235 is multilevel or hierarchical mixed models. The Behavioral Statistics 235 are run on Pre-Existing Data 237. The Behavioral Statistics 235 determine, for example, what kinds of environmental risk variables are related to specific EMA responses (such as drug craving) and what duration of environmental exposure most reliably predicts EMA responses. After the Behavioral Statistics 235 produce an outcome, the results of the outcome are used to guide the Machine Learning 239 analyses, as discussed below

The Machine Learning 239 is used to develop an automated inference for a future EMA response. The automated inference is based on Pre-Existing data 241 that are used to develop and test a training model. For example, the Behavioral Statistics in operation 235 could show that environmental risks such as drug paraphernalia on the sidewalk contribute to heroin craving, and that exposure to these risks 6 hours prior to the EMA entry are good predictors of EMA reports of craving. The machine learning 239 would then be set up to use the 6 hours of exposure data prior to an EMA response. To develop a future-predicting model, for example, at 30, 60 or 90 minutes into the future, the specific amount of time prior EMA entry is dropped from the 6 hours of data. Meaning, if the intent of the machine-learning model 239 is to predict heroin craving 90 minutes into the future with the environmental-exposure data, environmental-exposure increments representing time between 0 and 90 minutes before the event are dropped from the full exposure sample. Rather, if 6 hours of time are to be used to predict heroin craving 90 minutes into the future, these predictions would use environmental-exposure data between 91-420 minutes prior to the EMA event. The output of the machine learning 239 is a new model (stored as a new self-contained file) that infers an EMA-derived outcome from the Pre-Existing 241 data where the results are at an acceptable accuracy, such as a kappa greater than 0.6. Under these definitions for a machine-learning model 239 at an acceptable accuracy, we can use Pre-Existing data 241 to make an inference about New Data 221 without having to re-train the model every time New Data are transferred into the system. In step 243, as New Data are transferred 27, 121 into the system, the New Data are processed 221. The output at 231 is then fed into the Machine Learning 239 output, which is the saved files from the machine learning model derived from Pre-Existing data 209 and which is at an acceptable accuracy. The New Data 245, which are real-time data in some instances, are then run with the output from 239 to predict a future EMA response, for example, at increments of 30, 60, and 90 minutes into the future. The output from the prediction or inference is an automated decision 247 about a future event. In one embodiment, the automated decision 247 is a new number or output that is stored as a text file (i.e., txt, csv, etc.) and is transferred 249 back to the mobile device 253 in a similar capacity as described above. During the data transfer, the mobile device 253 is in use by the user in the natural environment 251. If the automated decision 247 indicates a statistical likelihood that the user will experience a negative behavioral event in the near future, for example, heroin craving 30, 60, or 90 minutes into the future, the mobile device 253 will automatically instigate an intervention 255. The specific mode of mobile-based intervention can vary, from a vibrating device, a sound, a flashing screen on the mobile device 253, to a recommendation that the user use coping skills to reduce craving, leave the risky situation, or contact a source of support. In addition to an automated intervention, the user can also self-monitor their risk of a future negative event by checking the automated decision at their own discretion or at intervals throughout the day that are preset by the clinician. Thus, the mobile device 253 can raise the patient's awareness of risk and deliver the automated decision 247 in real time while the user is in the natural environment 251.

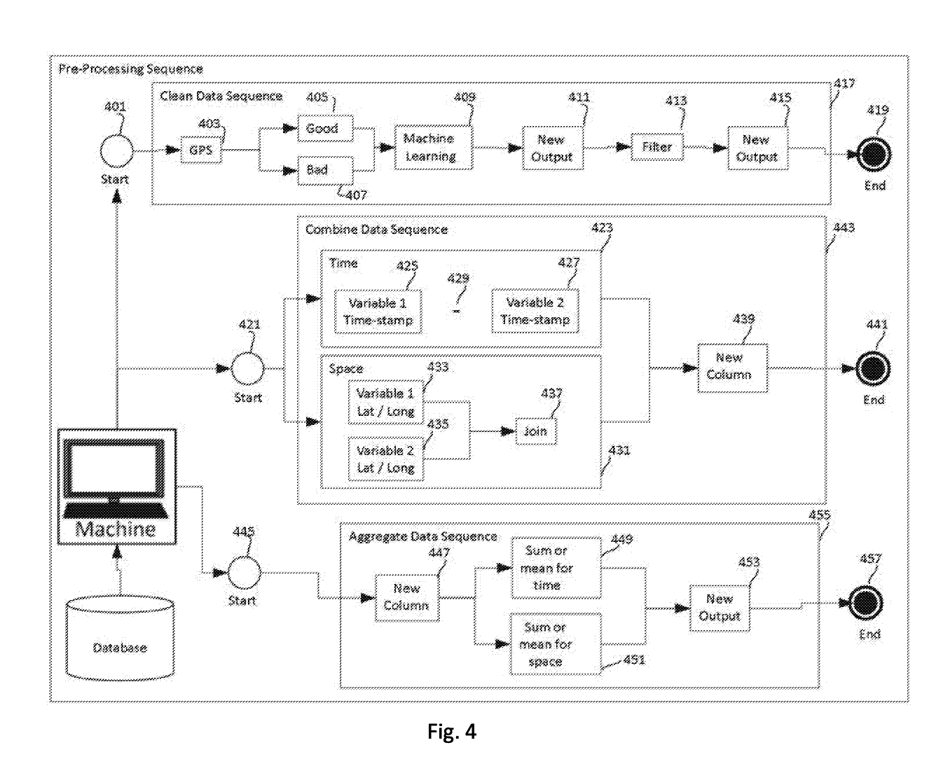

FIG. 4 is a schematic diagram illustrating a pre-processing sequence for a mobile health platform system. The illustration of FIG. 4 may be utilized by the system outlined above in relation to FIG. 3. In the embodiment shown, a Clean Data Sequence 417 starts 401 with new GPS data 403 collected from a user via a mobile device 17, 117. Raw GPS data 403 may have location error related to satellite signal quality. Thus, the mobile health system on machine 7 may utilize internal data-quality-control calculations made by the software on the receiver to determine the quality of the raw GPS data 403. Sometimes the internal quality controls fail to be calculated, which leaves a certain proportion of GPS data 403 with no quality control. First, GPS data 403 with quality-control measures are used and classified as good 405 data or bad 407 data. The GPS data 403 thus classified are then transmitted to machine-learning software 409, which is used to develop a predictive model with ancillary features in the GPS data such as altitude, distance, speed, and time. Once a new machine-learning model 409 is developed to a reasonable accuracy, the model is then implemented on GPS data with no quality-control values to classify them as good or bad. The new outputs 411 from the scoring are then used to remove potentially bad data. Next, a signal-processing filter 413 is run on the good GPS data 405 or 411 to further improve coordinate location. One example of a filter 413 that may be utilized in this process is a Kalman filter. The filtering 413 smooths longitude and latitude by timestamp and velocity recorded at each timestamp. A separate filter is run on each track recorded of GPS locations per user. The filtering 413 can also infer coordinates that are considered low quality by the machine learning flagging for good and bad quality data. In general, filtering 413 is an optional component to the process. The result of the filtering 413 is a New Output 415 for longitude and latitude per GPS recorded location, which is the end 419 of the Clean Data Sequence.

A Combine Data Sequence 443 may also be included or performed that starts 421 optionally with either combining the data over Time 423 or Space 431. Combining over Time 423 may be used with a Time Stamp for Variable 1 425, normally GPS and/or mobile device data from 415, to be combined mathematically with the Time-Stamp for Variable 2 427. Variable 2 can be any other data stored in the Database 123 as long as the data has a Time-Stamp. For example, Clinical Data 125 are recorded when a user is in a Clinic, and normally in less frequent periods than the GMA/GPS data. The Clinical Data 125 can be joined to GPS/GMA data that are recorded at more frequent intervals than the Clinical Data. Time-Stamp may include a unique value representing time that can be compared mathematically to another value. To combine Variable 1 and Variable 2, the Time-Stamp of Variable 2 is subtracted 429 from the Time-Stamp of Variable 1. The result of the subtraction 429 is a New Column 439 indicating the difference in time between Variable 1 and Variable 2. The Combine Data Sequence for Space 431 uses a latitude (i.e., Lat) and a longitude (i.e., Long) for Variable 1, which is the GPS/GMA data from 415, to Join 437 to Variable 2. Variable 2 435 is any Map Data 129 stored on the Database 123. The Join 437 of Variable 1 and Variable 2 includes GPS/GMA location coordinates being used to sample vector or raster data. The result of the Join 437 is a New Column 439 indicating the value from Variable 2 for each coordinate value Variable 1. Creating the New Column 439 is the End 441 of the Combine Data Sequence 443.

An Aggregate Data Sequence 455 is a processing step implemented to simplify the results of the Combine Data Sequence 443 for data output in the New Column 439, which is included in the Aggregate Data Sequence as 447. The Aggregate Data Sequence 455 is executed to convert these new raw numbers in 447 (linked to the GPS/GMA data on a row-by-row basis) to new numbers that can be processed through statistics and predictive analytics. This can be utilized when the GPS sampling is either i) too dense to discern any meaningful behavioral statistic or prediction (i.e., one replicate a minute per day) or ii) randomly varying in time, where the randomness reduces any numerical inference obtained from the behavioral statistics and predictive analyses for consistent replication in time. There are generally two ways to aggregate the new data 447, either by time or by space. In operation 449, aggregating by time is completed by either summing or averaging 447 the data to consistent units of time. For example, data 447 can be in any increment greater than a minute and/or used to create a consistent replication in time from randomly varying temporal data. If the Environmental Risk Maps 15, 111 are sampled in 437, output 439 is a new column with unique measure for each Environmental Risk Map at the precise latitude and longitude for each coordinate recorded by the GPS/GMA/mobile device. The time-stamps of the GPS/GMA/mobile device can then be used to aggregate 449 the unique environmental risk values per coordinate to consistent average values for the same unit in time. For example, a new column with a disorder map sampled to every GPS time stamp could be aggregated to average disorder value per 10, 20 or 30 minutes. In operation 451, the new column of data 447 is either averaged or summed over space. For example, if the space data from 431 are joined to vector files, such as a boundary polygon representing a neighborhood map, the result in column 439 is a unique value per neighborhood for each coordinate collected by the GPS/GMA. The corresponding GPS/GMA and/or Environmental Risk Maps can then be aggregated as the sum or average of any of these values 451 per neighborhood. The result of either 449 or 451 is a New Output 453 for either the data aggregated by time or space.

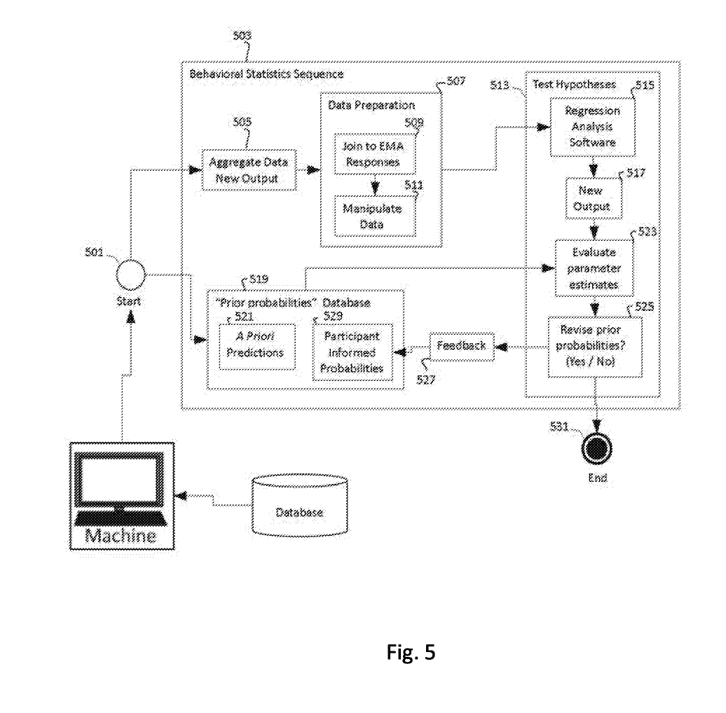

FIG. 5 is a schematic diagram illustrating a behavioral statistics sequence for a mobile health platform system. In one embodiment, the behavioral statistics sequence 503 is the same or similar behavioral statistics sequence 235 described above with reference to FIG. 3. The Behavioral Statistics Sequence 235, 503 is an automated database learning system that is coupled to behavioral-statistics software. The process starts with the outputs of the aggregate data sequence 455, shown in FIG. 5 as box 505. The Data Preparation 507 step is used here to either join 509 EMA responses 21, 117 to the aggregated data 505 and/or complete additional processing for data manipulation 511. In box 509, the EMA responses are joined to the aggregate data output 505 by adding the responses as new columns in output 505. To do this, output 505 may be first transposed from columns to rows as part of the joining process 509. The Data Manipulation 511 completed here on the aggregate data output 505 adjusts the output joined with EMA responses 509 to specifically fit the input-parameter requirements for the behavioral-statistics software 515. Additionally, output 505 joined to responses 509 can include additional data manipulation 511 to create specific time-series analyses, such as assessing the cumulative experience of environmental exposure (from environmental mapping 111, 129) prior to an EMA event logged by the mobile device 117. For example, cumulative environmental exposures of up to twelve hours or more prior to the event logging in the mobile device 17 may be used to assess the relationship between environmental exposures at varying cumulative increments and the response in the GMA. This may be necessary because the behavioral-statistics software is not designed to run test statistics in time-series, so the temporal element in the data must be first manipulated to account for and normalize exposure values for time.

The Test Hypotheses processing sequence 513 is used to test whether information collected by the mobile device 17 on users' EMA responses 21 remains consistent with a priori predictions 521 as new cohorts of users are evaluated. Regression analysis software is used to assess the relationship between users' EMA responses 21 and any other data stored on the database 123 and processed through the Pre-Processing Sequences 207 shown in FIG. 4 (417, 443, 455). An example here is an Environmental Risk Map 15 stored on the database 9, processed through processes 417, 443, and 455, and then through process 507. It is after all the data processing through step 507 that the data from the Environmental Risk Map 15 may be quantitatively compared to the EMA responses. The regression model used here falls generally under the term multilevel model, which can also be called any of the following: hierarchical model, mixed model, or random-effects model. The data output from data preparation process 507 is thus plugged into regression analysis software 515 and the result is new output 517. New output 517 can be any assortment of test-statistics describing the relationship between EMA responses and any other data stored on the database 123—typically an odds ratio, a beta weight, or a group of model-adjusted means, all with associated confidence intervals.

The Behavioral Statistics Sequence 503 may also include the Prior Probabilities Database 519. The database 519 contains empirically based estimates of the likelihood that a given new hypothesis is true, or that a parameter will assume some given range of values. After each run of the regression analysis software 515 and the generation of New Output 517 for parameter values, an automated code to Evaluate Parameter Estimates 523 may be executed. Here, the processing initially compares output 517 to the a priori predictions 521. The next step is to determine whether to Revise the Prior Probabilities 525. In one particular embodiment, a zero or low value in operation 523 means no and a one or positive value in operation 523 means yes. If the Revise Prior Probabilities 525 determines a no, the process ends 531. Alternatively, if the Revise Prior Probabilities 525 is determined to be a yes, then there is internal feedback 527 to the Prior Probabilities Database 519. As new data enter the Behavioral Statistics Sequence from the aggregate data output 505, data preparation 507, and test hypothesis process 513, the processing is replicated, where evaluating parameter estimates 523 is tested against either a priori predictions 521 and/or user informed probabilities 529. User informed probabilities 529 can be updated for test statistics as more and more user data enter the Behavioral Statistics sequence, which may cause the revise prior probabilities decision 525 to change in light of more data and/or as new EMA responses are uploaded.

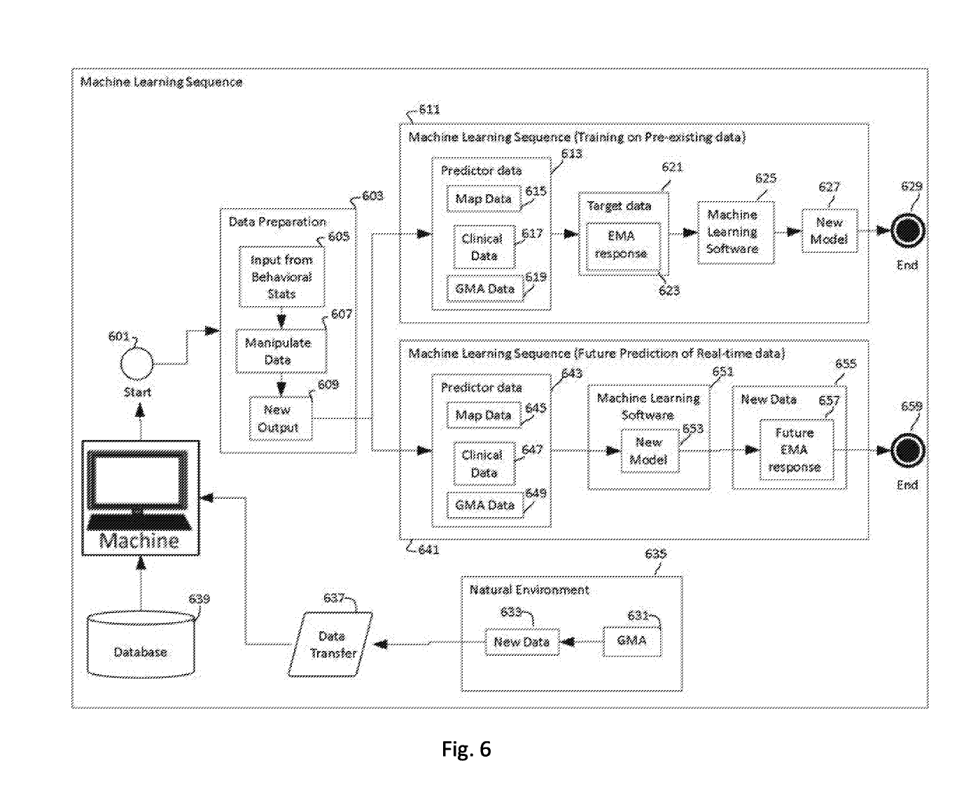

FIG. 6 is a schematic diagram illustrating a machine learning sequence for a mobile health platform system. The process starts 601 with a Data Preparation process 603, where Input from the Behavioral Statistics 605 sequence is used to Manipulate Data 607 from output 453 and/or aggregate data new output 505. The combination of input from behavioral stats 605 and the manipulated data 607 means that input data to the machine learning are used for data that are found to have robust predictive value in the test statistics from output 517. One example is the calculation of a time window for significant relationships between environmental risk data and EMA responses. For example, some studies show significant relationships between environmental risk exposures and subjective responses up to 6 hours later. Informed by those findings, the Machine Learning Sequence would subset into a New Output 609 only data for the 6 hours immediately preceding each EMA time stamp. However, any subset of the data may be utilized by the sequence. In addition to this, when time-series-based predictions are used, the point is to predict a future response. In order to run these future predictions, for example at 30, 60 or 90 minutes into the future, data 30, 60 or 90 minutes in time immediately preceding the EMA time stamp are dropped from the time-series sample used to make the prediction. This additional data manipulation is part of operation 607 to output 609.

Output 609 from the data preparation is used as the input to the Machine Learning 239, which is broken into two sequences. The first sequence is illustrated as operation 239 of FIG. 3 and deals with running the Machine Learning Sequence on Pre-existing data 611 on the database. Predictor data 613 shows the possible data inputs that can be used as Predictor data, which include, but are not limited to: Map Data 615, Clinical Data 617, and GMA Data 619. Predictor data 613 is used to predict the Target data 621 for any EMA Response 623. Predictor data 613 and target data 621 can be at any increment in time prior to the EMA event (i.e., 30, 60 or 90 minutes into the future). Machine-learning software 625 is used to develop a training model that uses predictor data 613 to predict target data 621. Machine learning software 625 can be, for example, a regression or classification predictor. Examples of machine-learning software 625 are support-vector machines, random Forests, and adaboost. The output from machine-learning software 625 is a new file that includes the newly developed machine-learning model 627, allowing the system, as shown in FIG. 1, to make automated predictions.

As shown in FIG. 1, Data Transfer 27 may occur between the mobile device 17 in the Natural Environment 13 and the machine 7 in the clinical setting 3. FIG. 2 also shows the data transfer in 121 between the Natural Environment 113 and the machine 107 of the clinical setting 101 in database development. Additionally, FIG. 3 shows how a Future Prediction 243 is made with New Data 245 in the Data Analytics 233 sequence. In FIG. 3, future prediction 243 shows this processing of real-time data to make future predictions, where process 243 is described in further detail in operation 641 of FIG. 6. First, to get new real-time data, the mobile device 631 collects new data 633 in the natural environment 635. The mobile device transfers data through data transfer 637 to the machine 7. In general, the data are Pre-Processed through the sequences outlined above in relation to FIG. 5, and then through the data preparation operation 603, so that it replicates the same data format and structure as was used in predictor data 613. The Machine-Learning Sequence for Future Prediction of Real Time data is shown in sequence 641. Here predictor data 643 is the same or similar to predictor data 613, except the processing uses new, real-time data in predictor data 643. The new model 653 created in the machine-learning software is then accessed with the machine-learning software 651 and processed with predictor data 643. The output is a new data file 655, which has been scored for a future EMA prediction 657, for example, the EMA value at 30, 60, or 90 minutes into the future.

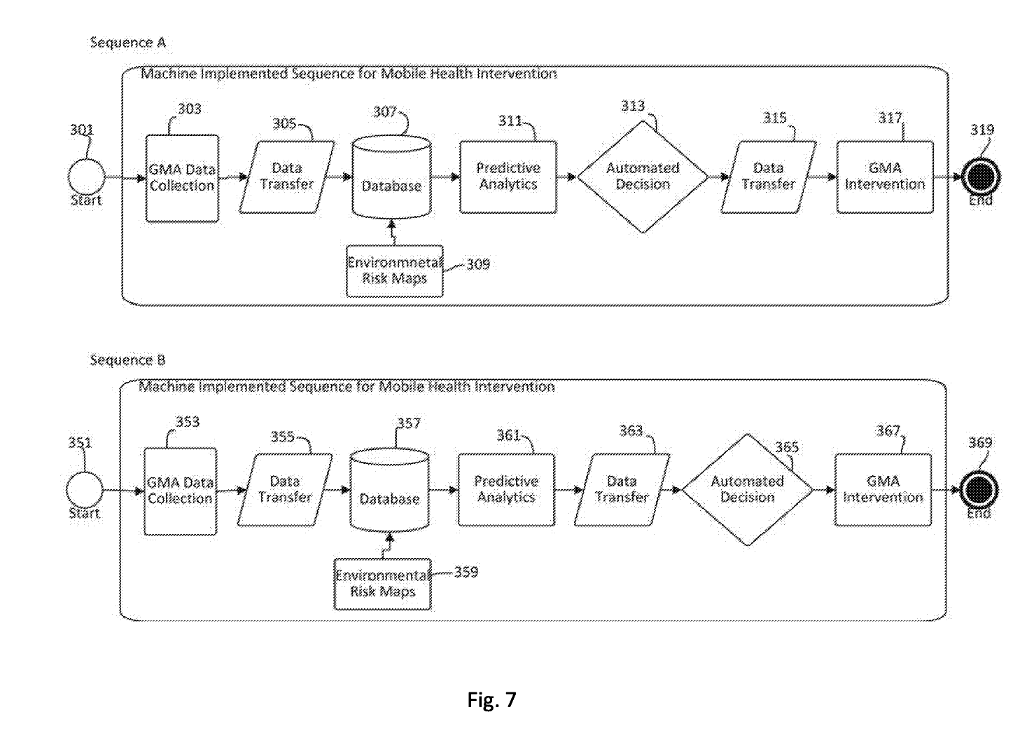

In review, FIG. 7 illustrates a machine-implemented sequence for a mobile health intervention that is delivered as one attribute of the Mobile Health Platform. Sequence A starts at 301, where a mobile device unit automatically collects data in operation 303. Further detail is provided on operation 303 in FIG. 1 utilizing the mobile device 17 (with GPS component 19 and EMA component 21), self-reporting data 23, and intensive ambulatory physiological monitoring components 25 and in FIG. 2. Returning to FIG. 7, the GMA data are transferred 305 back to the database 307 where they are combined with environmental-risk maps 309 that also reside in the database. Further descriptions of data transfer 305, database 307, and environmental risk maps 309 are provided in machine 7, database 9, and environment risk management 15 of FIG. 1 and machine 107, database 109, environmental mapping 111, and data transfer 121 of FIG. 2. In another operation of sequence A, predictive analytics (i.e., machine learning or data mining) 311 are run on the contents of the database to generate an automated decision 313. Additional descriptions for predictive analytics operations 311 and automated decision 313 are provided in data processing and analytics 29 and automated decision 31 of FIG. 1, pre-processing operations 207 and data analytics operations 233 of FIG. 3 and in the content related to FIGS. 4, 5 and 6. After an automated decision is generated 313, it is transferred back to the mobile unit 17 in operation 315 and an automated intervention is triggered by the mobile unit in operation 317 if the user is at imminent risk of a future negative event. Further descriptions of steps data transfer 315 and mobile or GMA device intervention 317 are provided in steps data transfer 27, mobile device 17, and intervention 33 of FIG. 1 and operations 249-255 in FIG. 3. Sequence B replicates Sequence A, except in that the data transfer 261 occurs before the automated decision in step 263. In relation to FIG. 3, Sequence B in FIG. 7 means that steps 243 and 245 can occur on the mobile device 253 and come after step 249. Some individual contents of the sequence can be omitted, changed, edited, adapted, or implemented as one full automated step.

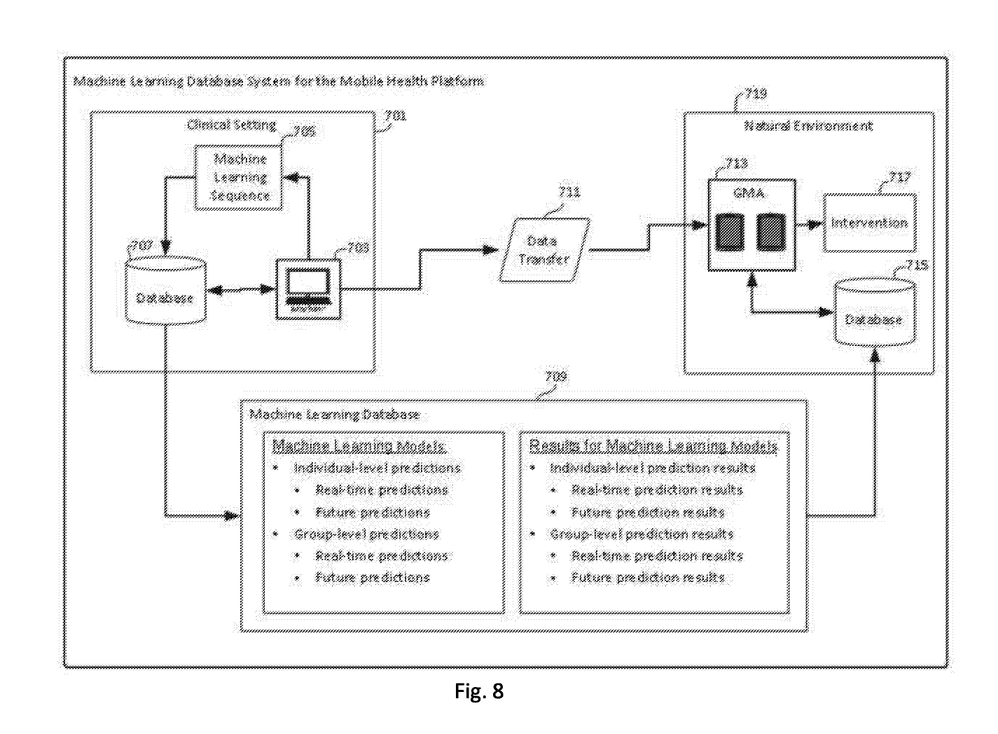

FIG. 8 is a schematic diagram illustrating a machine learning model database for a mobile health platform system. The system is based on a machine 703 that may be in a clinical setting 701. At the end of the Machine Learning Sequence in 705, a new model 627 and a new prediction 655 are saved in a database in 707, 611, and 641. Database 707 is the Machine Learning Database, which stores an assortment of machine learning models and their new prediction outputs. The contents of the Machine Learning Database are shown in more detail in box 709 in the flowchart. Box 709 shows that the database is split into two parts 1) machine learning models and 2) the output from machine learning models: new predictions. The models can predict for either an individual person or a group of 2 or more people in any combination that yields an accurate model. This process can be used for either real-time momentary predictions or future predictions at any point into the future. Momentary predictions mean a prediction for the GMA at its present local time. A future prediction is a prediction result for an event at a specified increment beyond the GMA's present time, for example, 30, 60 or 90 minutes into the future. The machine learning database also stores the results to the machine learning models. When there is a data transfer to the GMA 711, 27, and 249, the data stored on the Machine Learning Database 707 are transferred to 715, which is the Machine Learning Database on the handheld mobile device 711, 17, 253 being carried by the user in the natural environment 719. Database 715 stores the models and outputs that are most accurate for the user carrying that mobile or GMA device 713. This means different GMA devices could store different machine learning models or results in database 715, but all models and results are stored in database 707 on a machine 709. Depending on the processing capabilities of the GMA, the data stored in database 715 could be either output 627 or output 655. If the GMA device does not have the capacity to process data and the machine learning model, then output 655 is stored in database 715. If the GMA device does have the capacity to process data and the machine learning model, then output 627 is stored on database 715. The GMA 713 accesses database 715 to determine whether to initiate an intervention 717. Database 707 can be stored in any kind of machine 703 that can be used to continuously update database 707 via data transfer 27, 121, 637, which stores all data from all the GMA devices in the system. In addition, database 707 can be separated into multiple databases and/or integrated, updated, or merged with any of the databases described in FIGS. 1-7. The machine learning database in database 715 is only updated at regular increments in time (e.g., daily or weekly) when output 627 is stored on database 715. When output 655 is stored on database 715, the database is updated more frequently (e.g., every 10 mins, 20 minutes or 30 minutes, depending on data transfer rates in data transfer 711).

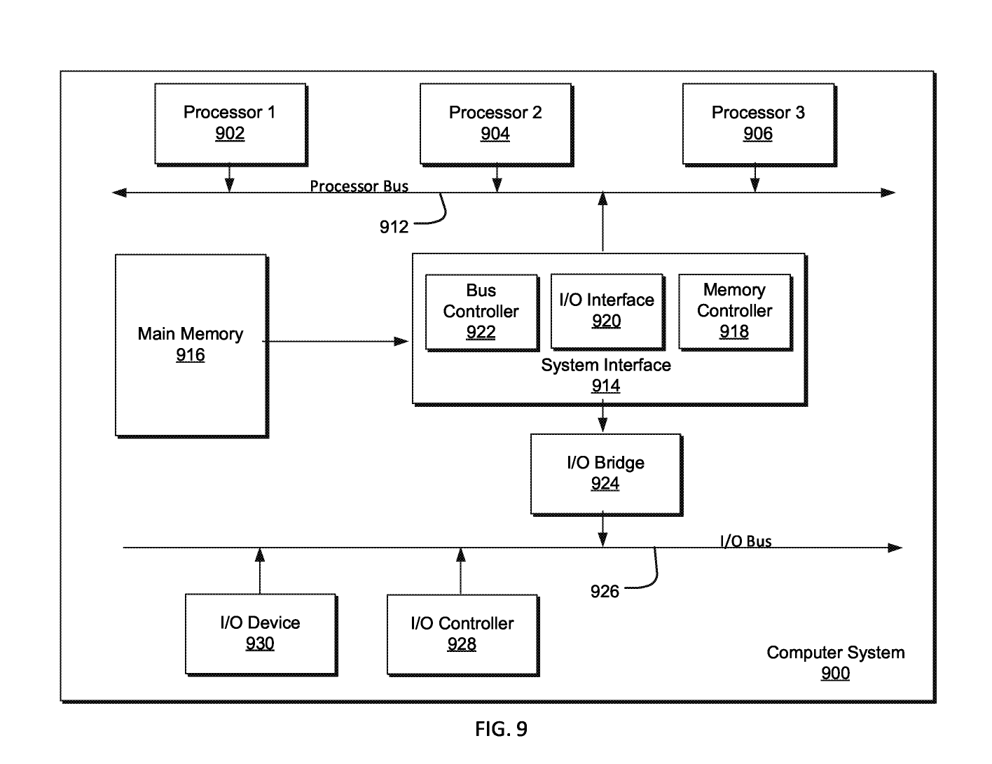

FIG. 9 is a block diagram illustrating an example of a computing device or computer system 900 which may be used in implementing the embodiments of the components of the network disclosed above. For example, the computing system 900 of FIG. 9 may be the machine 7 of the clinical setting 3 discussed above. The computer system (system) includes one or more processors 902-906. Processors 902-906 may include one or more internal levels of cache (not shown) and a bus controller or bus interface unit to direct interaction with the processor bus 912. Processor bus 912, also known as the host bus or the front side bus, may be used to couple the processors 902-906 with the system interface 914. System interface 914 may be connected to the processor bus 912 to interface other components of the system 900 with the processor bus 912. For example, system interface 914 may include a memory controller 914 for interfacing a main memory 916 with the processor bus 912. The main memory 916 typically includes one or more memory cards and a control circuit (not shown). System interface 914 may also include an input/output (I/O) interface 920 to interface one or more I/O bridges or I/O devices with the processor bus 912. One or more I/O controllers and/or I/O devices may be connected with the I/O bus 926, such as I/O controller 928 and I/O device 940, as illustrated.

I/O device 940 may also include an input device (not shown), such as an alphanumeric input device, including alphanumeric and other keys for communicating information and/or command selections to the processors 902-906. Another type of user input device includes cursor control, such as a mouse, a trackball, or cursor direction keys for communicating direction information and command selections to the processors 902-906 and for controlling cursor movement on the display device.

System 900 may include a dynamic storage device, referred to as main memory 916, or a random access memory (RAM) or other computer-readable devices coupled to the processor bus 912 for storing information and instructions to be executed by the processors 902-906. Main memory 916 also may be used for storing temporary variables or other intermediate information during execution of instructions by the processors 902-906. System 900 may include a read only memory (ROM) and/or other static storage device coupled to the processor bus 912 for storing static information and instructions for the processors 902-906. The system set forth in FIG. 9 is but one possible example of a computer system that may employ or be configured in accordance with aspects of the present disclosure.

According to one embodiment, the above techniques may be performed by computer system 900 in response to processor 904 executing one or more sequences of one or more instructions contained in main memory 916. These instructions may be read into main memory 916 from another machine-readable medium, such as a storage device. Execution of the sequences of instructions contained in main memory 916 may cause processors 902-906 to perform the process steps described herein. In alternative embodiments, circuitry may be used in place of or in combination with the software instructions. Thus, embodiments of the present disclosure may include both hardware and software components.

A machine readable medium includes any mechanism for storing or transmitting information in a form (e.g., software, processing application) readable by a machine (e.g., a computer). Such media may take the form of, but is not limited to, non-volatile media and volatile media. Non-volatile media includes optical or magnetic disks. Volatile media includes dynamic memory, such as main memory 916. Common forms of machine-readable media may include, but are not limited to, magnetic storage medium (e.g., floppy diskette); optical storage medium (e.g., CD-ROM); magneto-optical storage medium; read only memory (ROM); random access memory (RAM); erasable programmable memory (e.g., EPROM and EEPROM); flash memory; or other types of medium suitable for storing electronic instructions.

Embodiments of the present disclosure include various steps, which are described in this specification. The steps may be performed by hardware components or may be embodied in machine-executable instructions, which may be used to cause a general-purpose or special-purpose processor programmed with the instructions to perform the steps. Alternatively, the steps may be performed by a combination of hardware, software and/or firmware.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations together with all equivalents thereof.

What is claimed is:

1. A system for providing an intervention notice to a user of a mobile device, the system comprising:
a network communication port for receiving a transfer of data from a mobile computing device, the received data from the mobile computing device comprising at least one indication of a geographic location of a user of the mobile computing device;
a database configured to store the received data from the mobile computing device; and
a computing device comprising a processing device and a computer-readable medium with one or more executable instructions stored thereon, wherein the processing device of the computing device executes the one or more instructions to perform the operations of:
receiving environmental risk mapping information associated with the user of the mobile computing device;
executing predictive analytics on the environmental risk mapping information with the at least one indication of the geographic location of the user of the mobile computing device, the predictive analytics comprising a predicted behavior of the user of mobile computing device; and
transmitting an automated decision to the mobile computing device through the network communication port, the automated decision configured to cause the mobile computing device to generate an intervention indicator for the user of the mobile computing device to alter the predicted behavior of the user.

2. The system of claim 1 wherein the data from the mobile computing device further comprises self-reported information of the user of the mobile computing device.

3. The system of claim 1 wherein the environmental risk mapping information is obtained from a third party database.

4. The system of claim 1 wherein the processing device further executes the one or more instructions to perform the operations of:
receiving initial user data from the user of the mobile computing device; and
storing the initial user data in the database with the received environmental risk mapping information.

5. The system of claim 1 wherein the data from the mobile computing device further comprises ambulatory physiological monitoring information of the user of the mobile computing device.

6. The system of claim 1 wherein associations are detected between the environmental risk mapping information and an indication of the behavioral state of the user of the mobile computing device at a geographic location.

7. The system of claim 1 wherein the predicted behavior of the user of the mobile computing device is based on either a regression or classification machine-learning function between the environmental risk mapping information and the at least one indication of a geographic location of a user of the mobile computing device.

8. The system of claim 7 wherein the processing device further executes the one or more instructions to perform the operations of:
receiving feedback information from the user of the mobile computing device of the accuracy of the automated decision; and
adjusting the machine-learning model in response to the feedback information from the user.

9. The system of claim 1 wherein the at least one indication of the geographic location of the user of the mobile device comprises a plurality of geographic locations of the mobile computing device for a particular amount of time prior to the transfer of data form the mobile computing device.

10. A computer-implemented method for an automated assessment of the momentary status of a user, the method comprising:
receiving a transfer of data from a mobile computing device associated with a user through a network connection, the received data from the mobile computing device comprising at least one indication of a geographic location of the user;
storing an environmental risk mapping information and the received data from the mobile computing device in a database;
executing predictive analytics on the environmental risk mapping information with the at least one indication of the geographic location of the user to generate a future prediction for the status of the user based on a machine-learning model; and
transmitting an automated decision to the mobile computing device through the network, the automated decision configured to cause the mobile computing device to generate an intervention indicator for the user to alter the predicted status of the user.

11. The computer-implemented method of claim 10 wherein the data from the mobile computing device further comprises self-reported information of the user received from the mobile computing device.

12. The computer-implemented method of claim 11 wherein the machine-learning model comprises the environmental risk mapping information with the at least one indication of the geographic location of the user and the self-reported information of the user.

13. The computer-implemented method of claim 10 further comprising:
obtaining the environmental risk mapping information from a third party database.

14. The computer-implemented method of claim 10 further comprising:
receiving initial user data from the user; and
storing the initial user data in the database with the received environmental risk mapping information.

15. The computer-implemented method of claim 10 wherein the data from the mobile computing device further comprises ambulatory physiological monitoring information of the user obtained by the mobile computing device.

16. The computer-implemented method of claim 10 wherein the at least one indication of the geographic location of the user comprises a plurality of geographic locations of the mobile computing device for a particular amount of time prior to the transfer of data from the mobile computing device.

17. The computer-implemented method of claim 10 wherein the intervention indicator for the user comprises a text-based message transmitted to the mobile computing device.

18. One or more non-transitory tangible computer-readable storage media storing computer-executable instructions for performing a computer process on a machine, the computer process comprising:
receiving initial user data from a user of a human behavior intervention system;
storing the initial user data in a user database with an environmental risk mapping information obtained from a third party database;
receiving a transfer of data from a mobile computing device through a network connection, the received data from the mobile computing device comprising at least one indication of a geographic location of a user of the mobile computing device;
executing predictive analytics on an environmental risk mapping information with the at least one indication of the geographic location of the user of the mobile computing device, the predictive analytics comprising a predicted behavior of the user of mobile computing device; and
transmitting an automated decision to the mobile computing device through the network communication port, the automated decision configured to cause the mobile computing device to generate an intervention indicator for the user of the mobile computing device to alter the predicted behavior of the user.

19. The one or more non-transitory tangible computer-readable storage media of claim 18, wherein the predicted behavior of the user of the mobile computing device is based on either a regression or classification machine-learning function between the environmental risk mapping information and the at least one indication of a geographic location of a user of the mobile computing device.

20. The one or more non-transitory tangible computer-readable storage media of claim 18, wherein the computer process further comprises:
receiving feedback information from the user of the mobile computing device of the accuracy of the automated decision; and
adjusting the machine-learning model in response to the feedback information from the user.
